(19) United States
(12) Patent Application Publication
(10) Pub. No.: US 2022/0304863 A1
(43) Pub. Date: Sep. 29, 2022

(54) Dressing With Fluid Level Indication

(71) Applicant: KCI Licensing, Inc., San Antonio, TX (US)

(72) Inventors: Justin Alexander LONG, Lago Vista, TX (US); Christopher Brian LOCKE, Bournemouth (GB); Timothy Mark ROBINSON, Shillingstone (GB); Thomas Alan EDWARDS, Hampshire (GB)

(21) Appl. No.: 17/619,697

(22) PCT Filed: Jun. 11, 2020
(86) PCT No.: PCT/US2020/037277

§ 371 Date: Dec. 16, 2021

Related U.S. Application Data
(60) Provisional application No. 62/862,255, filed on Jun. 17, 2019.

(51) Int. Cl. A61F 13/00

(52) U.S. Cl. CPC A61F 13/00055; A61F 13/00029; A61F 13/00059

(57) ABSTRACT

A dressing for a wound can include a body layer formed from an absorbent material. The dressing can also include multiple wicking cores disposed within the body layer. Each of the multiple wicking cores can be configured to provide a visual indication at a different state of liquid absorption in the body layer.

### CROSS-REFERENCE TO RELATED APPLICATIONS

This application claims the benefit of priority to U.S. Provisional Application No. 62/862,255, filed on Jun. 17, 2019, which is incorporated herein by reference in its entirety.

### BACKGROUND

The present disclosure relates generally to wound dressings. Wound dressings can provide a sterile interface to guard a patient's wound from contamination during the healing process. Dressings may also absorb wound fluid and/or increase the rate that wound fluid is removed from a wound bed. During the healing process, wound fluid can build up within the dressing and/or within an absorbent article therein.

### SUMMARY

One implementation of the present disclosure is a dressing for a wound, according to some embodiments. The dressing can include a body layer. The body layer may have a wound facing side and an environment facing side. The body layer can include one or more perforations that can have a depth into and/or through the body layer. The one or more indicators may be located at least partially in the one or more perforations. The one or more indicators can have a wound facing end and an environment facing end. The indicator may include a wicking material and a dye. The wicking material can be configured to draw liquid from the body layer to the environment facing end of the indicator. The dye can be in contact with and/or proximate to the environment facing end of the indicator. The dye can be configured to change color or opacity in response to contact with liquid drawn by the wicking material. The color or opacity change can indicate a level of fullness of the liquid in the body layer.

Another implementation of the present disclosure is a dressing for a wound, according to some embodiments. The dressing can include a liquid impermeable cover and multiple sensors. The liquid impermeable cover can be configured to seal to skin surrounding the wound to define an inner volume. The multiple sensors can be positioned within the inner volume defined by the cover. The sensors may be configured to transfer aqueous fluid at a bottom end of the sensor to an upper end of the sensor. At least one of the sensors and/or the cover can include a visual indication portion configured to change color or opacity in response to the sensor transferring aqueous fluid from the bottom end to the upper end. The change of color or opacity can indicate a fluid level within the inner volume defined by the cover.

Another implementation of the present disclosure is a method for indicating a liquid level within a wound dressing, according to some embodiments. The method can include absorbing liquid within an inner volume of the wound dressing. The wound dressing can be any of the dressings described herein. The method can also include transferring the liquid through a wicking material to an upper end of the wicking material. The method can also include changing a color or opacity of at least one of the wicking material, a sensor portion adjacent the wicking material, and a portion of a wound drape corresponding to the wicking material, the color change occurring in response to contact with the liquid to indicate a liquid level within the wound dressing.

Another implementation of the present disclosure is a method for using any of the dressings described herein, according to some embodiments. The method can include providing the dressing over a wound and monitoring a visual indication of the dressing. The visual indication of the dressing can indicate an amount of fluid within the dressing.

Another implementation of the present disclosure is a method for using any of the dressings described herein, according to some embodiments. The method can include providing the dressing over a wound and fluidly coupling an inner volume of the wound and/or the dressing with a negative pressure wound therapy device. The method can include performing negative pressure wound therapy for the wound using the negative pressure wound therapy device.

Another implementation of the present disclosure is a dressing for a wound, according to some embodiments. The dressing can include a body layer formed from an absorbent material. The dressing can also include multiple wicking cores disposed within the body layer. One or more of the multiple wicking cores can be configured to provide a visual indication at a different state of liquid absorption in the body layer.

Another implementation of the present disclosure is a kit that includes components for assembling or forming any of the dressings described herein, according to some embodiments. The kit can include one or more negative pressure wound therapy devices or components. The one or more negative pressure wound therapy components or devices can be assembled to fluidly couple the dressing with a negative pressure source, such as a negative pressure wound therapy unit.

Those skilled in the art will appreciate that the summary is illustrative only and is not intended to be in any way limiting. Other aspects, inventive features, and advantages of the devices and/or processes described herein, as defined solely by the claims, will become apparent in the detailed description set forth herein and taken in conjunction with the accompanying drawings.

### BRIEF DESCRIPTION OF THE DRAWINGS

FIG. 1 is an exploded view of a wound dressing with fluid level indication, according to some embodiments.

FIG. 2 is a side view of the wound dressing of FIG. 1, according to some embodiments.

FIG. 3 is a top view of the wound dressing of FIG. 1, according to some embodiments.

FIG. 4 is a side view of the wound dressing of FIG. 1 with fluid reaching a first indicator, according to some embodiments.

FIG. 5 is a side view of the wound dressing of FIG. 1 with fluid reaching a first and second indicator, according to some embodiments.

FIG. 6 is a top view of the wound dressing of FIG. 1 with fluid at a first level, according to some embodiments.

FIG. 7 is a top view of the wound dressing of FIG. 1 with fluid at a second level activating a first indicator, according to some embodiments.

FIG. 8 is a top view of the wound dressing of FIG. 1 with fluid at a third level activating a second indicator, according to some embodiments.

FIG. 9 is a top view of the wound dressing of FIG. 1 with fluid at a fourth level activating a third indicator, according to some embodiments.

FIG. 10 is a top view of the wound dressing of FIG. 1 with fluid at a fifth level activating a fourth indicator, according to some embodiments.

FIG. 11 is an exploded perspective view of the wound dressing of FIG. 1, according to some embodiments.

FIG. 12 is an exploded perspective view of the wound dressing of FIG. 11, according to some embodiments.

FIG. 13 is a side section view of the wound dressing of FIG. 11, according to some embodiments.

FIG. 14 is a side view of one of the indicators of the wound dressing of FIG. 1, according to some embodiments.

FIG. 15 is a side view of one of the indicators of the wound dressing of FIG. 1, according to some embodiments.

FIG. 16 is a top view of one of the indicators of the wound dressing of FIG. 1, according to some embodiments.

FIG. 17 is a top view of one of the indicators of the wound dressing of FIG. 1, according to some embodiments.

### DETAILED DESCRIPTION

### Overview

Referring generally to the FIGURES, a dressing that provides visual indications of fill level is shown. Advantageously, the dressing facilitates managing wound exudate and notifying the caregiver when the dressing is near full. The dressing can include a patient or wound interface layer, a body layer, one or more indicators, and a barrier or cover layer.

The wound interface layer can be a perforated layer. The wound interface layer can in some instances include a silicone or polyurethane (PU) gel interface layer. The wound interface can be a hydrophilic foam layer (if a silicone perforated wound interface layer is not used) which readily absorbs exudate in the dressing. The wound interface together with the barrier layer can form a hybrid drape adhesion system. If the wound interface layer is not used, then the body layer can be used as the wound interface (e.g., contacts the wound, is applied to the wound, etc.).

The body layer can be a hydrophilic, highly wicking foam interface layer, an absorbent foam, and/or an absorbent layer. The body layer can include a polyurethane foam. The body layer can be white in color so that exudate coloration can be easily viewed by a caregiver. The body layer can have a degree of fluid retention such that wound exudate is held in place in the body layer. The body layer may be a superabsorbent layer encapsulated by wicking layers on either sides to increase fluid retention capacity of the dressing. The body layer may have holes punched therethrough or partially through at positions where the indicators are placed. Wicking layers making up either side of the body layer can be welded together in a tea-bag at the edges and also in areas where the body layer has been punched to form vacancies for the indicators to occupy.

The indicators may be integrated into the body layer and may be removable (e.g., no adhesive, welding, tacking, etc., used to retain the indicator in place) or non-removable. The indicators can be vertically oriented. The indicators can include a portion that is a wicking material. The indicators can be located just below or at an upper surface of the body layer, and extend through the body layer at different depths. Therefore, as fluid level within the dressing reaches different depths, the indicators provide a visual indication showing the fluid level within the dressing. The indicators can also be at locations that start at different depths from the upper surface of the body layer and extend through the body layer a different depths or the same depth. The indicators can also be at locations that start at the same upper surface or same depth from the upper surface and extend through the body layer at the same depth or through the entire body layer. In these instances, the indicators can optionally contain different amounts of wicking and spacing material (e.g., wicking materials to different depths in different indicators from a plane of the upper surface of the body layer) to enable the indicators to provide a visual indication showing different fluid levels within the dressing. The wicking material of the indicators can be composed of a range of suitable materials (e.g., viscose, polyester, cellulose, etc.) to facilitate transport of fluid from a surface of the wound, from the body layer, and/or from a depth within the body layer to or near the upper surface of the body layer and/or to the interior surface of the barrier layer. The indicators or wicking materials may be removable and/or replaceable to facilitate collecting wound exudate samples. Hydrochromic dye, such as hydrochromic ink may be printed onto the barrier layer and/or on to an upper surface of the indicator. The hydrochromic dye can activate, change color, change opacity, etc., in response to a liquid fluid, such as an aqueous fluid and/or a wound exudate, to provide a visual indication to the caregiver regarding the fluid level within the dressing.

The barrier layer can seal with skin surrounding a patient's wound and define an inner volume between the barrier layer and the patient's skin, wound, and/or wound bed. The barrier layer can provide a protective layer that prevents contamination yet is breathable and allows evaporation. The barrier layer can contain a suitable adhesive for secure fixation of the dressing.

A super absorbent material sheet may add to the fluid handling capacity of the dressing and provides additional fluid retention properties. The body layer can also be or contain an absorbent layer and/or absorbents.

As liquid fluid is absorbed into the body layer and/or the indicators, it can be wicked up the indicators when the liquid fluid contacts the bottom of the wicking material of the indicator. The wicking material can wick the liquid fluid up to the hydrochromic dye/ink. The hydrochromic dye/ink may activate upon contact by the fluid to provide a visual indication to the caregiver regarding a fill level in the dressing. Advantageously, this facilitates notifying the caregiver when the dressing may need to be changed before the dressing becomes full of fluid. In some instances, the transparent barrier layer in combination with the body layer, being white or a light color, facilitates visualization of the exudate color and/or the activated hydrochromic dye/ink.

The dressing described herein can also provide a visual indication of remaining dressing fluid capacity, which can facilitate appropriate dressing changes. For example, a dressing that reaches maximum fluid capacity can be removed. Likewise, the caregiver can visually determine that the dressing is not yet full and may not remove the dressing. Therefore, the dressing can improve caregiver efficiency and reduces the likelihood that a caregiver will change a dressing too early (e.g., before the dressing is full) or too late (e.g., after the dressing has become full).

The dressing described herein can be used as a stand-alone dressings, or in conjunction with a negative pressure wound therapy system and/or instillation system. For example, the negative pressure wound therapy system and/or instillation system may inject or introduce a sterile solution to the dressing to facilitate sterility and healing of the wound.

### Wound Dressing

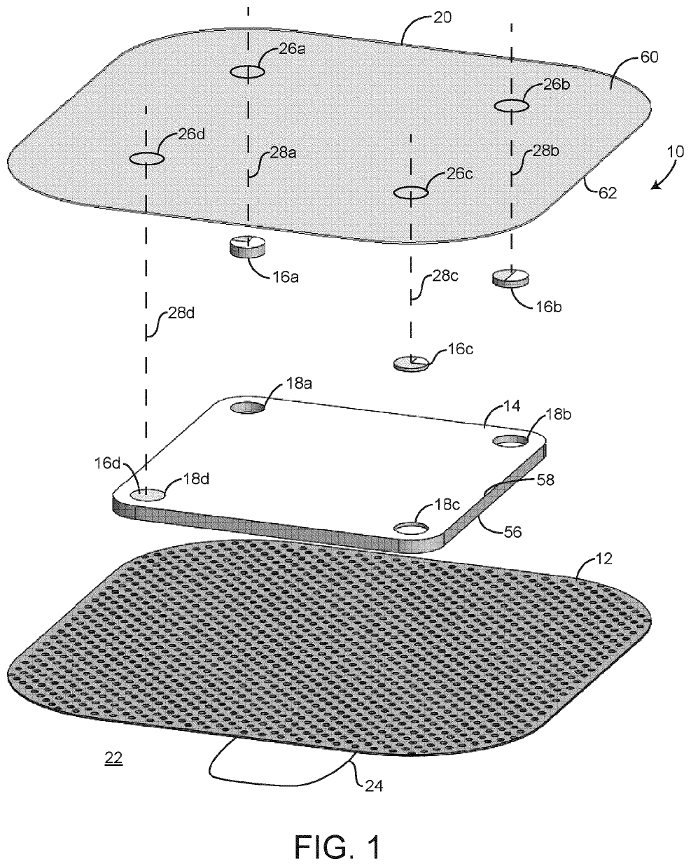

Referring particularly to FIG. 1, a wound dressing, a dressing, a bandage, etc., shown as wound dressing 10 can include a first layer, a bottom layer, a wound interface layer, a perforated layer, etc., shown as patient interface layer 12, according to some embodiments. Patient interface layer 12 may be configured to engage and directly seal with a patient's skin 22 surrounding a wound 24. Patient interface layer 12 can be a perforated wound/patient contact layer. Patient interface layer 12 may be optional. Patient interface layer 12 can be a perforated layer that contains silicone or a polyurethane gel elastomer.

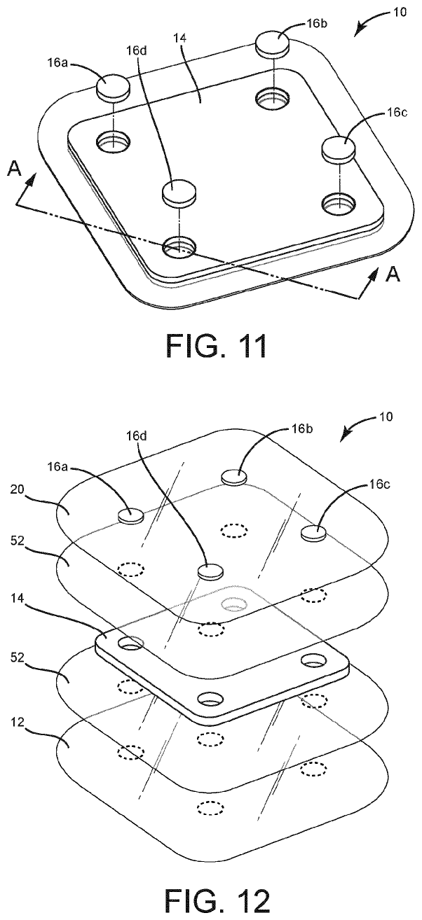

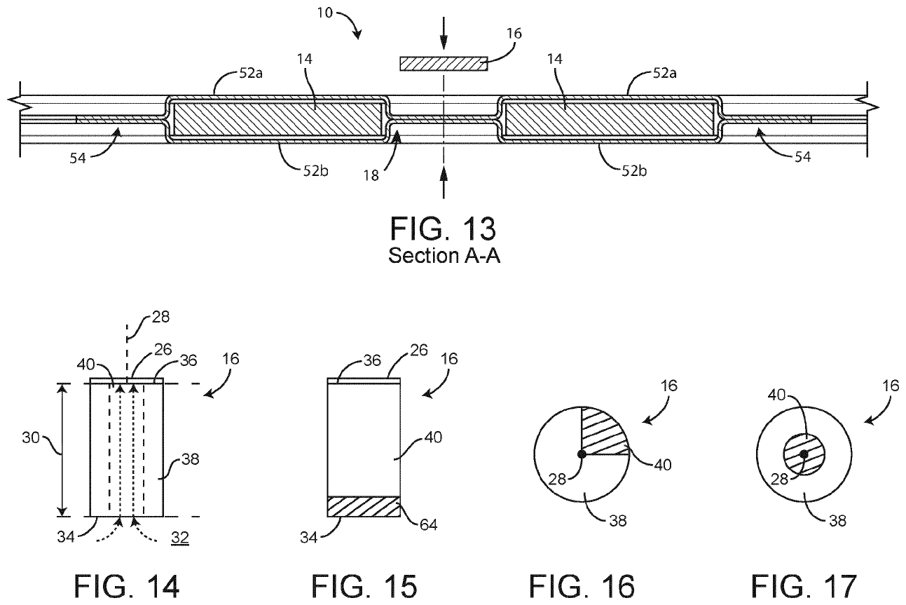

Wound dressing 10 can also include a foam layer, a body layer, a foam interface layer, etc., shown as body layer 14, according to some embodiments. Body layer 14 can be a hydrophilic, highly wicking foam material, a plastic, ABS, an absorbent layer, etc. Body layer 14 can also include a polyurethane foam. Body layer 14 may be white colored or light in color so that exudate coloration can be easily viewed. Body layer 14 can have a degree of fluid retention such that wound exudate from wound 24 is held in place. Body layer 14 can be replaced with a superabsorbent encapsulated by wicking layers on either side (shown in FIGS. 11-13). If patient interface layer 12 is not used, body layer 14 may directly engage or be placed over wound 24. Body layer 14 can include a wound facing surface, side, face, etc., shown as wound facing side 56. Wound facing side 56 may be directed towards or facing wound 24. Body layer 14 can also include an environment facing surface, side, face, etc., shown as environment facing side 58. Environment facing side 58 may be opposite wound facing side 56.

Body layer 14 includes apertures, recesses, grooves, holes, bores, cylindrical cut-outs, inner volumes, channels, slots, etc., shown as apertures 18. In some embodiments, apertures 18 are disposed along a perimeter of body layer 14. Apertures 18 can be positioned along one or more edges of body layer 14, or may be positioned at a central location of body layer 14, or in any pattern or location of body layer 14. Apertures 18 are configured to receive corresponding cores, wicking cylinders, cylindrical members, tubular members, wicking cores, sensors, dipsticks, wicks, indicators, etc., shown as indicators 16. In some embodiments, apertures 18 are configured to receive indicators 16 therewithin. A lower end of indicators 16 can be disposed within the corresponding aperture 18, while an upper or top end of indicators 16 may be flush or even with or even with the environment facing side 58 of body layer 14. In other embodiments, the upper or top end of indicators 16 is sub-flush with the environment facing side 58 of body layer 14. In some embodiments, the environment facing side 58 of body layer 14 is substantially parallel with the wound facing side 56 of body layer 14 that can directly engage or contact the patient's skin 22. Apertures 18 are each associated with a corresponding axis 28. Axis 28 extends normally through a centerpoint of the corresponding aperture 18. Axes 28 can be substantially parallel to each other. For example, axis 28a corresponds to aperture 18a and indicator 16a, axis 28b corresponds to aperture 18b and indicator 16b, axis 28c corresponds to aperture 18c and indicator 16c, and axis 28d corresponds to aperture 18d and indicator 16d.

Wound dressing 10 can also include an upper layer, an outer most layer, a wound drape, a thin film, a flexible layer, a liquid impermeable layer, a flexible covering, etc., shown as barrier layer 20. Barrier layer 20 can be configured to substantially cover and contain body layer 14, indicators 16, and patient interface layer 12. Barrier layer 20 can define an inner volume of dressing 10 therewithin. Barrier layer 20 can be clear, transparent, translucent, opaque, colored and transparent, selectably opaque (e.g., that transitions between opaque and transparent in response to fluid), etc. Barrier layer 20 can be positioned on top of body layer 14 and may circumferentially enclose body layer 14 and patient interface layer 12. Barrier layer 20 can include a suitable adhesive for secure fixation of barrier layer 20. Barrier layer 20 can include an adhesive coated along an interior or bottom surface, shown as wound facing side 62. Barrier layer 20 also includes an exterior surface, an exterior side, an outer side, etc., shown as environment facing side 60. Indicators 16 can be viewable through barrier layer 20. Barrier layer 20 may be breathable, but also provide a protective layer to prevent contamination while allowing evaporation.

The adhesive can be configured to adhere to the patient's skin 22. Barrier layer 20 can be configured to substantially cover body layer 14, indicators 16 and may be capable of attaching to and/or being sealed to tissue surrounding wound 24. The wound facing side 62 of barrier layer 20 can include adhesive over an entirety of the surface, or along a perimeter of barrier layer 20 (e.g., along the edges of barrier layer 20), or in a pattern of the surface.

Fluid or liquid may build up within the inner volume defined by barrier layer 20. The fluid (e.g., wound fluid, saline solution, etc.) can be absorbed by a bottom end of indicators 16 and may travel upwards along indicators 16 until it contacts barrier layer 20. In some embodiments, the fluid is absorbed and transferred by different indicators 16 based on a fill level of fluid within the inner volume defined by barrier layer 20. Barrier layer 20 can include fluid-activated ink, fluid-activated dye, hydrochromic ink, etc., shown as ink 26. In some embodiments, ink 26 is printed on the interior or inner surface (e.g., wound facing side 62) of barrier layer 20. The interior or inner surface of barrier layer 20 may be directly adjacent or in contact with an outer or upper layer of body layer 14 when wound dressing 10 is fully assembled. In some embodiments, ink 26 is positioned on body layer 14 such that ink 26 is directly in contact with or adjacent to corresponding indicators 16 therebelow. In some embodiments, the ink 26 is positioned on the environmental side (upper side) of the indicators 16 so that the ink 26 is activated when fluid that is wicked through the indicators contacts and activates the ink 26. In some embodiments, a water-soluble dye and/or ink is positioned internal to indicators 16 so that fluid flow to the environmental side of the indicators 16 through the wicking material of the indicators 16 brings the water-soluble dye and/or ink to the environmental side of the indicators 16 so that it can be visualized.

For example, ink 26a can be positioned or printed on the interior surface of barrier layer 20 such that ink 26a is in direct engagement or contact with indicator 16a when wound dressing 10 is fully assembled. Likewise, ink 26b, 26c, and 26d may be positioned or printed on the interior surface of barrier 20 such that ink 26b, 26c, and 26d are in direct engagement or contact with indicators 16b, 16c, and 16d, respectively, when wound dressing 10 is fully assembled. In some embodiments, an entirety of the interior surface of barrier layer 20 is coated with ink 26.

Indicators 16 can be integrated into body layer 14. In some embodiments, indicators 16 are cylindrical members that are vertically oriented (e.g., oriented along axes 28). Indicators 16 can be positioned and spaced along edges of body layer 14, or may be positioned at a central location of body layer 14, or in any pattern or location of body layer 14. Indicators 16 can be formed from (e.g., extruded, co-extruded, formed, etc.) a hydrophilic material. Indicators 16 can be made from or include cellulose acetate, cellulose, polyester, viscose, cotton, a non-woven material, a fibrous material, etc., herein referred to as a “wicking material.” The wicking material can be made of a material that is suitable for transporting or absorbing fluid. The wicking material can facilitate the transportation of fluid from the bottom or lower end (the wound facing side) of indicators 16 to an upper or top end (the environmental facing side) of indicators 16. Indicators 16 can be completely composed of the wicking material, or may be partially composed of the wicking material. For example, indicators 16 can include a sector that is formed of the wicking material (e.g., a quarter of a circle, a half of a circle, etc.), a portion that is formed of the wicking material (e.g., a top quarter of the indicator 16, a top half of the indicator 16, etc.), or be completely made of a wicking material, etc.

In some embodiments, dressing 10 is usable in a negative pressure wound therapy application. For example, dressing 10 can include a manifold, a coupling portion, a coupler, an attachment device, etc., that facilitates fluid coupling between the inner volume of dressing 10 defined by barrier layer 20 and a negative pressure wound therapy device/unit. The coupler can facilitate removably and/or fluidly coupling a tubular member with the inner volume of dressing 10.

Dressing 10 can be provided to a caregiver in a kit. The kit can include any of the components of dressing 10 described herein (e.g., negative pressure wound therapy components, patient interface layer 12, body layer 14, indicators 16, barrier layer 20, etc.). The caregiver can assemble dressing 10 to form dressing 10 as shown in the FIGURES.

Referring particularly to FIG. 14, one of indicators 16 is shown in greater detail, according to some embodiments. Indicators 16 can each include wicking material 40 and support material 38. In some embodiments, wicking material 40 and support material 38 are co-extruded. In other embodiments, indicator 16 is completely formed of wicking material 40. Wicking material 40 can form a sector of indicator 16 (e.g., a sector of a circle, a core of indicator 16, etc.) and is configured to extend through substantially an entire height 30 of indicator 16. Wicking material 40 can form a portion of the indicator 16 height 30 (e.g., a top quarter of the indicator 16, a top half of the indicator 16, etc.) and is configured to extend through substantially an entire width of indicator 16 where the wicking material is located. Height 30 can vary across different indicators 16. For example, a first indicator 16 may have a first overall height 30, a second indicator 16 may have a second overall height 30 smaller than the first overall height 30, a third indicator 16 may have a third overall height 30 smaller than the second overall height 30, and a fourth indicator may have a fourth overall height 30 smaller than the third overall height 30 (e.g., the smallest indicator). In some instances, support material 38 is spacing material between the bottom surface of the wicking material 40 and the bottom surface of the indicator 16. The spacing material can be a material other than wicking material 40. In some embodiments, the spacing material is a water-absorbent material. In some embodiments, the spacing material is not a water absorbent material.

Wicking material 40 is configured to facilitate the transfer or transportation of fluid along substantially the entirety of height 30 of indicator 16 or a portion of the height 30 of indicator 16. For example, wicking material 40 can facilitate the transfer or transportation of fluid from a bottom surface, a lower surface, a bottom end, a lower end, a bottom side, a lower side, etc., shown as lowermost surface 34 of indicator 16 or a lowermost surface of the wicking material 40 within the indicator 16 that can be above the lowermost surface 34 of the indicator (not shown) to a top surface, top face, top side, top end, environment facing end, environment facing side, environment facing face, environment facing surface, etc., shown as upper most surface 36 of indicator 16. In this way, once fluid 32 reaches bottom surface 34 of indicator 16 or bottom most surface of the wicking material 40, fluid 32 is transferred or transported upwards along the entire height 30 of the wicking material 40, which can in some instances be the entire or a portion of the entire height 30 of indicator 16 to upper surface 36 of indicator 16. Once fluid 32 reaches upper surface 36 of indicator 16, fluid 32 may activate a dye, a fluid activated dye, etc., thereby causing the dye to change color or opacity to indicate a fluid level in dressing 10. In other embodiments, a water-soluble dye or fluid activated dye is present within wicking material 40 such that as fluid 32 is transferred or transported through wicking material 40, the color or opacity of wicking material 40 changes to indicate fill level (or a level of fullness) in dressing 10 (or in body layer 14).

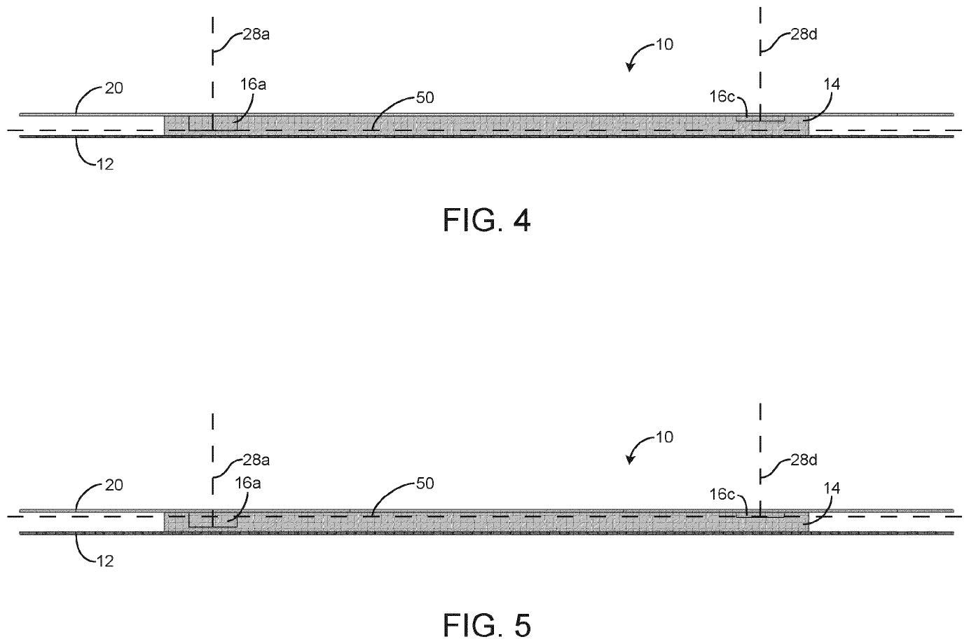

Referring particularly to FIG. 15, a lower portion, a lower end, a bottom portion, a bottom end, etc., of indicator 16 shown as bottom portion 64 can be support material 38 or a spacing material. The support material 38 or spacing material can be an absorbent material or a non-absorbent material. The support material 38 or the spacing material at bottom portion 64 of indicator 16 can be a material that is different than wicking material 40. Bottom portion 64 can have a same cross-sectional shape as indicator 16. In some embodiments, bottom portion 64 defines a portion of indicator 16. For example, bottom portion 64 can have a height that is a portion of the entire height 30. Bottom portion 64 can define any portion of indicator 16 (e.g., half of indicator 16, a quarter of indicator 16, an eighth of indicator 16, etc.). Portions of indicator 16 other than bottom portion 64 can be or include wicking material 40 and/or support material 38. The indicator 16 may wick fluid or liquid to upper most surface 36 in response to a fill level or level of fullness of fluid within dressing 10 (e.g., fluid level 50 as shown in FIGS. 4 and 5) exceeding or filling above a height of bottom portion 64. In this way, a height of bottom portion 64 can vary for indicators 16a-16d such that indicators 16a-16d wick liquid to upper most surface 36 in response to changing or different fluid levels 50.

Referring to FIGS. 14 and 15, dye 26 can be positioned at upper most surface 36 of indicator 16. In some embodiments, dye 26 is directly adjacent (e.g., in contact with) upper most surface 36. Dye 26 can be activated to change color or opacity in response to fluid (e.g., a liquid fluid) transferred or wicked through wicking material 40 of indicator 16. In some embodiments, dye 26 is directly adjacent or in contact with wicking material 40 of indicator 16.

Referring particularly to FIGS. 16 and 17, indicator 16 can have a circular cross-sectional shape. It should be understood that indicator 16 can have a square cross-sectional shape, an elliptical cross-sectional shape, a hexagonal cross-sectional shape, a generally circular cross-sectional shape, etc., or any other cross-sectional shape. Wicking material 40 can be a portion of the overall cross-sectional shape of indicator 16. For example, wicking material 40 may be a sector of the circular cross-section of indicator 16 as shown in FIG. 17. Specifically, FIG. 17 shows indicator 16 including a quarter of a circle that is wicking material 40, with the other portions of the cross-sectional shape of indicator 16 being formed with support material 38. Likewise, wicking material 40 can form a core of indicator 16 as shown in FIG. 17 and may be surrounded with support material 38. In any case, in some embodiments, wicking material 40 extends from bottom surface 34 of indicator 16 to upper surface 36 of indicator 16 to facilitate the transfer and/or transport of fluid 32 from bottom surface 34 to upper surface 36 of indicator 16. In some embodiments, wicking material 40 is removable and/or replaceable so that wicking material 40 can be easily removed and replaced.

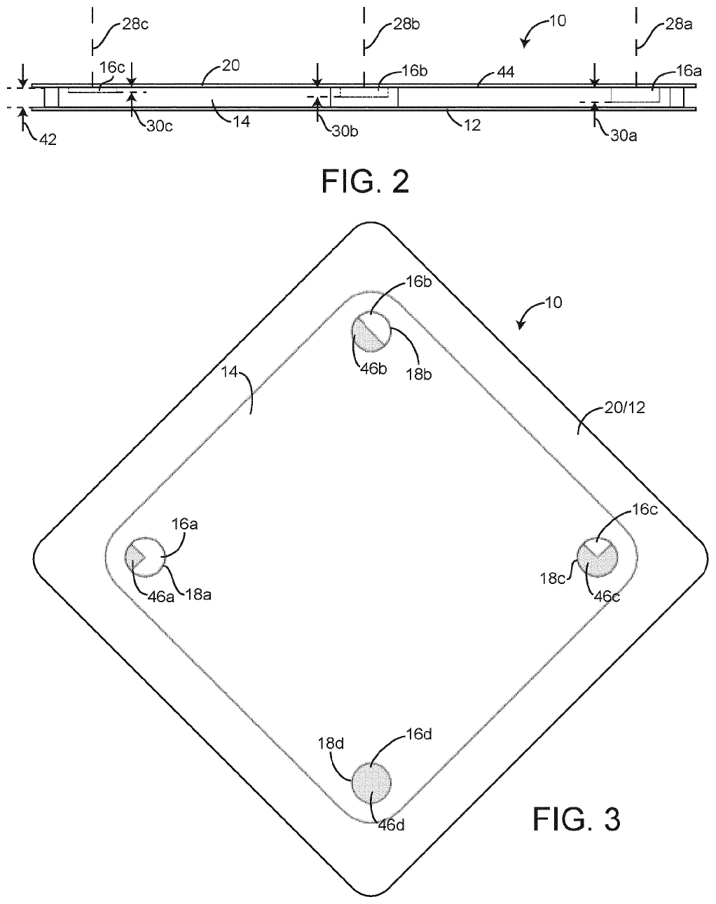

Referring particularly to FIG. 2, a side view of dressing 10 is shown, according to some embodiments. FIG. 2 shows indicator 16a, indicator 16b, and indicator 16c extending through body layer 14. Each indicator 16 has a different height 30 corresponding to a different fill level or degree of fill of dressing 10. Specifically, indicator 16a has a height 30a that is approximately three quarters of an overall height 42 of body layer 14, indicator 16b has a height 30b that is half of overall height 42, and indicator 16c has a height 30c that is one quarter of overall height 42. Indicator 16a has height 30a that extends from an upper or top surface 44 of body layer 14 to a position within body layer 14. Specifically, height 30a extends from upper surface 44 of body layer 14 to a position that is three quarters or seventy five percent of overall height 42 of body layer 14. Likewise, indicator 16b has height 30b and extends from upper surface 44 of body layer 14 halfway into body layer 14. Indicator 16c has height 30c and extends from upper surface 44 of body layer 14 a quarter into body layer 14.

Upper surface 36 of indicators 16 may each be substantially flush or co-planar with upper surface 44 of body layer 14. In this way, if barrier layer 20 is positioned on top of body layer 14, upper surface 36 of indicators 16 can each directly engage or be in contact with barrier layer 20. Similarly, upper surface 44 of body layer 14 may be in direct contact or directly engage barrier layer 20. If ink 26 is printed onto the interior or inner surface of barrier layer 20, upper surfaces 36 of indicators 16 may directly engage or be in direct contact with ink 26 of barrier layer 20 when dressing 10 is assembled. In some instances, upper surface 36 of indicators 16 are not substantially flush or co-planar with upper surface 44 of body layer 14; however, the upper surface 36 of indicators 16 can still be visible in a top down view of the dressing in this embodiment. In this way, the indicators 16 can all be the same height, but be located at different depths within the body layer 14.

The varying heights 30 or depths of indicators 16 results in indicators 16 drawing or wicking fluid 32 from bottom surface 34 of indicators 16 and/or bottom surface of the wicking material 40 to upper surface 44 of the body layer 14 and/or upper surface 36 of the indicator 16 in response to changes in fluid or fill level in dressing 10. For example, once dressing 10 is a quarter or 25 percent filled with fluid 32, indicator 16a may draw fluid from bottom surface 34 to upper surface 44. Likewise, once dressing 10 is half full or 50 percent filled with fluid 32, indicator 16b may draw fluid from bottom surface 34 to upper surface 44. Once dressing 10 is three quarters or 75 percent filled with fluid 32, indicator 16c may draw fluid from bottom surface 34 to upper surface 44. Finally, indicator 16d may have a negligible height such that once fluid 32 fills substantially the entirety of dressing 10, fluid is drawn from bottom surface 34 of indicator 16d to upper surface 44 of indicator 16d. Once fluid is drawn from bottom surface 34 of indicators 16 or bottom surface of the wicking material to upper surface 44 of one of indicators 16, the fluid may activate corresponding ink 26. In some embodiments, ink 26 is configured to change color or opacity in response to fluid or liquid. In this way, the fill level of fluid within dressing 10 can be visually indicated as dressing 10 fills. Advantageously, ink 26 and/or upper surface 36 of indicators 16 can be visually inspected by a caregiver to easily determine a fill level of dressing 10. The caregiver can easily identify which of indicators 16 the corresponding ink 26 is activated to determine a corresponding fill level. Ink 26 can be different colors, shapes, etc., to indicate a degree of fill of fluid within dressing 10. This facilitates notifying a caregiver when dressing 10 should be changed or flushed.

In some embodiments, a portion of indicators 16 is formed from wicking material 40. In other embodiments, multiple section of indicators 16 are formed from wicking material 40. For example, a first section or sector of indicators 16 can be formed from wicking material 40 having a first height 30, while a second section or sector of indicators 16 can be formed from wicking material 40 having a second height 30 where the second height 30 is greater than the first height 30. In some embodiments, the multiple sections of wicking material 40 of indicators are separated by support material 38 and/or spacing material. In this way, the multiple sections of wicking material 40 can independently absorb the fluid and draw the fluid to upper surface 36. In some embodiments, ink 26 is associated with each of the multiple sections of wicking material 40. The various ink 26 can activate in response to the fluid reaching bottom surface of each of the multiple sections of wicking material 40. In this way, a single indicator 16 can provide a visual indication for the various fill levels of the fluid within dressing 10.

Referring particularly to FIG. 3, a top view of dressing 10 is shown, according to some embodiments. Dressing 10 includes indicators 16a-16d. Indicators 16a-16d are configured to be received within corresponding apertures 18 of body layer 14. Indicators 16a-16d can have various heights 30 of wicking material 40 such that indicators 16a-16d absorb, transfer, or transport fluid from bottom surface 34 of bottom surface of the wicking material 40 (which may be the same as or different than the bottom surface 34 of the indicator) to upper surface 44 in response to the fill level of fluid within dressing 10 reaching various predetermined levels. For example, indicator 16a can have a height 30 such that indicator 16a or wicking material 40 extends three quarters of the way through body layer 14 from upper surface 44, indicator 16b can have a height 30 such that indicator 16b or wicking material 40 extends halfway through body layer 14 from upper surface 44, indicator 16c can have a height 30 such that indicator 16b or wicking material 40 extends a quarter of the way through body layer 14, and indicator 16d can have a height 30 such that indicator 16d or wicking material 40 extends a very small amount through body layer 14 from upper surface 44.

Indicators 16 can each provide a visual indication 46 that shows a caregiver a fill level of dressing 10. The shape, color, size, etc., of visual indications 46 can vary depending on indicators 16 to indicate different degrees of fill of dressing 10. For example, indicator 16a may correspond to visual indication 46a indicating that dressing 10 is a quarter full, indicator 16b may correspond to visual indication 46b that dressing 10 is half full, indicator 16c may correspond to visual indication 46c that dressing 10 is three quarters full, and indicator 16d may correspond to visual indication 46d that dressing 10 is filled with fluid. Visual indications 46 can consecutively activate (e.g., change color, change opacity, etc.) in a progressive manner response to the corresponding indicators 16 absorbing fluid from bottom surface 34 and transferring or transporting the fluid to upper surface 36.

In some embodiments, visual indications 46 are the corresponding ink 26. Visual indications 46 can show the pattern that ink 26 is printed onto the interior surface of barrier layer 20. For example, visual indication 46a shows a quarter of a circle, indicating that dressing 10 is a quarter full, visual indication 46b shows half a circle, indicating that dressing 10 is half full, visual indication 46c shows three quarters of a circle, indicating that dressing 10 is three-quarters full, and visual indication 46d shows a full circle, indicating that dressing 10 is full of fluid. As dressing 10 is filled with fluid (e.g., wound fluid, saline solution, etc.) over time, the various indicators 16 wick fluid and progressively activate visual indications 46 (e.g., from a quarter full to half full, etc., to completely full) to visually notify a caregiver regarding the fill level of dressing 10 (e.g., as the fluid within dressing 10 increases).

In some embodiments, indicator 16a is configured to change color or opacity to indicate that body layer 14 is filled to a quarter of a maximum capacity with liquid. In some embodiments, three quarters of indicator 16a is wicking material 40. Indicator 16b can be configured to change color or opacity to indicate that body layer 14 is filled to half of the maximum capacity. Half of indicator 16b can be wicking material 40. Indicator 16c can be configured to change color or opacity to indicate that body layer 14 is filled to three quarters of the maximum capacity with liquid. A quarter of indicator 16c may be wicking material 40. In this instance, indicators 16a-16d extend to the same or substantially the same depth into body layer 14.

In some instances, indicators 16a-16d extend to different depths into body layer 14. In some embodiments, indicator 16a is configured to change color or opacity to indicate that body layer 14 is filled to a quarter of a maximum capacity with liquid. In some embodiments, one quarter of the cross-section of indicator 16a is wicking material 40. Indicator 16b can be configured to change color or opacity to indicate that body layer 14 is filled to half of the maximum capacity. Half of the cross-section of indicator 16b can be wicking material 40. Indicator 16c can be configured to change color or opacity to indicate that body layer 14 is filled to three quarters of the maximum capacity with liquid. Three quarters of the cross-section of indicator 16c may be wicking material 40. Indicator 16d can be configured to change color or opacity to indicate that body layer 14 is filled to full of the maximum capacity with liquid. Substantially the entire cross-section of indicator 16d may be wicking material 40.

Referring particularly to FIGS. 4 and 5, dressing 10 is shown at various fill levels, according to some embodiments. As fluid fills in dressing 10, fill level 50 increases. Fill level 50 represents the upper surface of fluid within dressing 10. FIG. 4 shows the case when dressing 10 is a quarter full, whereby fluid reaches bottom surface 34 of indicator 16a. FIG. 5 shows the case when dressing 10 is three quarters full, whereby fluid reaches bottom surface 34 of indicator 16a and 16c. Once fill level 50 reaches bottom surface 34 of indicator 16a, the fluid is absorbed through bottom surface 34 of wicking material 40 and transferred/transported through wicking material 40 to upper surface 36 of indicator 16a. The fluid can then activate ink 26a that corresponds to indicator 16a, thereby providing a visual indication to a caregiver that dressing 10 is a quarter full. Likewise, once fill level 50 reaches bottom surface 34 of indicator 16c, the fluid is absorbed through bottom surface 34 of wicking material 40 of indicator 16c and transferred/transported through wicking material 40 to upper surface 36 of indicator 16c. The fluid can then activate ink 26d that corresponds to indicator 16c, thereby providing a visual indication to the caregiver that dressing 10 is three quarters full. In this way, indicators 16 provide a visual indication of fill level 50 which is viewable by the caregiver through barrier layer 20.

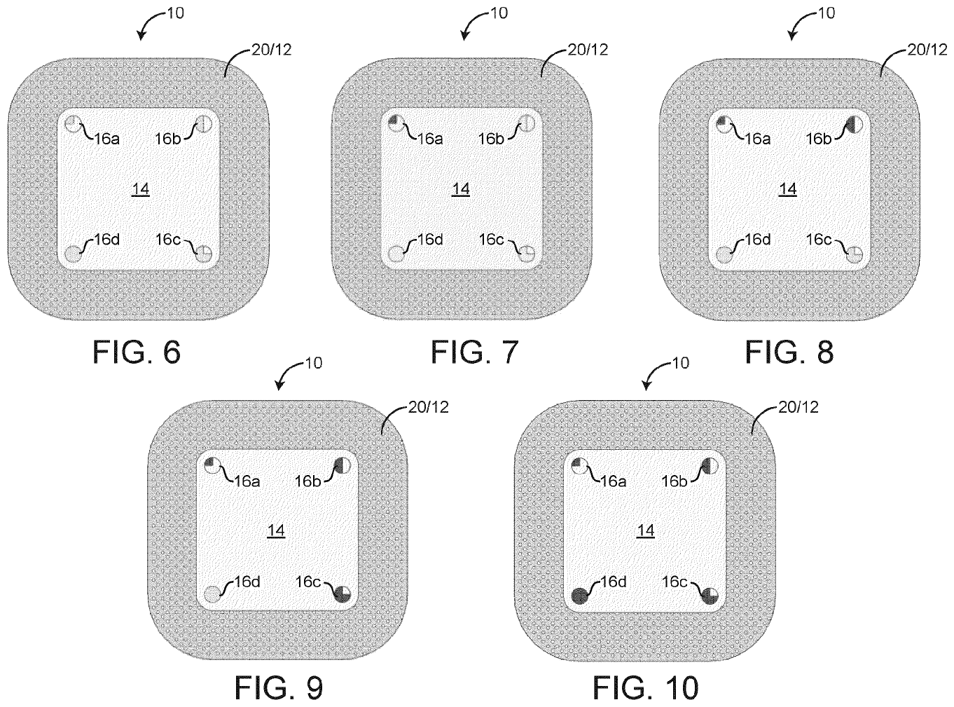

Referring particularly to FIGS. 6-10, dressing 10 is shown at various fill levels, according to some embodiments. FIG. 6 shows none of indicators 16 activated. Indicators 16 can remain de-activated if fill level 50 within dressing 10 is less than a predetermined amount (e.g., less than a quarter fully). Once fluid fills in dressing 10 such that fill level 50 reaches bottom surface 34 of indicator 16a or bottom surface of the wicking material of indicator 16a (e.g., once dressing 10 is at least a quarter or 25% full), indicator 16a transports the fluid to upper surface 36 and a visual indication is provided to a caregiver (e.g., with ink 26a, by changing a color, by changing opacity of barrier layer 20, etc., as shown in FIG. 7). Once fluid fills in dressing 10 such that fill level 50 reaches bottom surface 34 of indicator 16b or bottom surface of the wicking material of indicator 16b (e.g., once dressing 10 is at least half or 50% full), indicator 16b transports the fluid to upper surface 36 and a second visual indication is provided to the caregiver (as shown in FIG. 8). Similarly, once fluid fills in dressing 10 such that fill level 50 reaches bottom surface 34 of indicator 16c or bottom surface of the wicking material of indicator 16c (e.g., once dressing 10 is at least three quarters or 75% full), indicator 16c transports the fluid to upper surface 36 and a third visual indication is provided to the caregiver (as shown in FIG. 9). Finally, once fluid fills in dressing 10 such that fill level 50 reaches bottom surface 34 of indicator 16d or bottom surface of the wicking material of indicator 16d (e.g., once dressing 10 is completely full or saturated), indicator 16d transports the fluid to upper surface 36 and a fourth visual indication is provided to the caregiver (as shown in FIG. 10).

In this way, indicators 16 facilitate providing a visual indication to the caregiver regarding fill level 50. In some embodiments, the visual indication is a change in color due to ink 26 activating in response to being provided with fluid (e.g., through wicking material 40). In some embodiments, the visual indication for each of indicators 16 is the same color (e.g., a change of color from yellow to red, from clear to red, from clear to blue, etc.). In some embodiments, different indicators 16 are associated with different colored visual indications. For example, the visual indication for indicator 16a may be a green color, the visual indication for indicator 16b may be a blue color, the visual indication for indicator 16c may be a yellow or amber color, and the visual indication for indicator 16d may be an orange or red color.

In some embodiments, barrier layer 20 is coated with a selectable opaque material. The selectably opaque material can be disposed along an interior or inner surface of barrier layer 20 and may activated in response to fluid being provided by one of indicators 16. For example, barrier layer 20 may be opaque when dry, but when fluid is transported to barrier layer 20 through indicators 16 (e.g., through wicking material 40), barrier layer 20 (or a portion of barrier layer 20 that is in contact with the fluid) becomes transparent or translucent. Once barrier layer 20 becomes dry again, barrier layer 20 may return to appearing opaque.

In some embodiments, when dry, barrier layer 20 appears opaque due to the scattering of light caused by the differential in refractive index between air in the pores of particles in the barrier layer 20 and that of the particle itself. To maximize the transparency effect, the refractive index of the fluid and the particles of barrier layer 20 should be as close as possible. The refractive index of the fluid may be within about plus or minus five percent of the refractive index of barrier layer 20. In some embodiments, barrier layer 20 may be substantially within a range of about 1.0 to about 2.0. This may include, but is not limited to, refractive indices for barrier layer 20 (e.g., a selectively transparent layer) of about 1.0, 1.1, 1.2, 1.3, 1.4, 1.5, 1.6, 1.7, 1.8, 1.9, and 2.0, or any range there between. In some embodiments, the refractive index of barrier layer 20 may be substantially within a range of about 1.2 to about 1.75. In some embodiments, the refractive index of barrier layer 20 may be substantially within a range of about 1.33 to about 1.6.

Advantageously, barrier layer 20 can transition between the transparent state and the opaque state in response to the fluid. For example, if fluid level 50 within dressing 10 decreases, barrier layer 20 can return to the opaque state. In this way, a caregiver can easily monitor fluid level 50 within dressing 10 by performing a visual inspection.

It should be understood that any number of indicators 16 and corresponding ink 26 may be used with dressing 10. For example, only two indicators 16 may be used, with the first indicator 16 indicating when dressing 10 is half full, and the second indicator 16 indicating when dressing 10 is completely full of fluid. Ink 26 can be printed onto the interior surface of barrier layer 20, infused throughout indicators 16 (e.g., distributed throughout wicking material 40), or positioned at an additional or top portion of indicators 16. In some embodiments, a portion of indicators 16 and/or barrier layer 20 includes ink 26 or any other fluid activated material that changes color, opacity, shape, size, etc., in response to the fluid. For example a portion of indicators 16 can consist of ink or hydrochromic dye in addition to wicking material 40.

Referring particularly to FIGS. 11-13, dressing 10 is shown, according to another embodiment. Dressing 10 can have a tea-bag form, and includes an additional pair of wicking layers 52 that are welded along an entire perimeter of body layer 14. Specifically, upper wicking layer 52a can be welded with bottom wicking layer 52b at welds 54. Welds 54 can extend along an outer perimeter of wicking layers 52 such that body layer 14 is substantially sealed within wicking layers 52. Body layer 14 can be replaced with or manufactured from a super-absorbent material in such an embodiment.

Wicking layers 52 facilitate the absorption and transportation of fluid through indicators 16. For example, fluid may be absorbed through bottom wicking layer 52b and transferred to indicators 16 (or to wicking material 40 of indicators 16). Wicking layers 52 can be manufactured from the same material as wicking material 40 or from a similar material. After the fluid is absorbed and transported through wicking material 40 of indicator 16, the fluid is absorbed and transferred/transported through upper wicking layer 52a. In some embodiments, indicator 16 is positioned above upper wicking layer 52a such that the fluid is absorbed or wicked through wicking material 40 of indicator 16 after being transported/wicked through upper wicking layer 52a. The fluid can then be provided to corresponding ink 26 to provide the visual indication, or can be provided to barrier layer 20 (e.g., if barrier layer 20 is selectably opaque/transparent) to change the opacity of transparency of barrier layer 20 and provide a visual indication to the caregiver regarding fill level 50 within dressing 10. Advantageously, if barrier layer 20 is manufactured from a selectably opaque/transparent material, or is coated along an interior surface with the selectably opaque/transparent material, barrier layer 20 can transition back to the opaque/non-transparent state in response to the fluid level within dressing 10 decreasing. If the fluid level within dressing 10 increases again, barrier layer 20 can transition back into the transparent/translucent state.

### Configuration of Exemplary Embodiments

As utilized herein, the terms “approximately,” “about,” “substantially,” and similar terms are intended to have a broad meaning in harmony with the common and accepted usage by those of ordinary skill in the art to which the subject matter of this disclosure pertains. It should be understood by those of skill in the art who review this disclosure that these terms are intended to allow a description of certain features described and claimed without restricting the scope of these features to the precise numerical ranges provided. Accordingly, these terms should be interpreted as indicating that insubstantial or inconsequential modifications or alterations of the subject matter described and claimed are considered to be within the scope of the disclosure as recited in the appended claims.

It should be noted that the term “exemplary” and variations thereof, as used herein to describe various embodiments, are intended to indicate that such embodiments are possible examples, representations, and/or illustrations of possible embodiments (and such terms are not intended to connote that such embodiments are necessarily extraordinary or superlative examples).

The term “coupled,” as used herein, means the joining of two members directly or indirectly to one another. Such joining may be stationary (e.g., permanent or fixed) or moveable (e.g., removable or releasable). Such joining may be achieved with the two members coupled directly to each other, with the two members coupled to each other using a separate intervening member and any additional intermediate members coupled with one another, or with the two members coupled to each other using an intervening member that is integrally formed as a single unitary body with one of the two members. Such members may be coupled mechanically, electrically, and/or fluidly.

The term “or,” as used herein, is used in its inclusive sense (and not in its exclusive sense) so that when used to connect a list of elements, the term “or” means one, some, or all of the elements in the list. Conjunctive language such as the phrase “at least one of X, Y, and Z,” unless specifically stated otherwise, is understood to convey that an element may be either X, Y, Z; X and Y; X and Z; Y and Z; or X, Y, and Z (i.e., any combination of X, Y, and Z). Thus, such conjunctive language is not generally intended to imply that certain embodiments require at least one of X, at least one of Y, and at least one of Z to each be present, unless otherwise indicated.

References herein to the positions of elements (e.g., “top,” “bottom,” “above,” “below,” etc.) are merely used to describe the orientation of various elements in the FIGURES. It should be noted that the orientation of various elements may differ according to other exemplary embodiments, and that such variations are intended to be encompassed by the present disclosure.

The hydrophobicity of a material may vary, but to be considered hydrophobic, generally the material can have an average contact angle with water of at least 90 degrees in some embodiments. To be considered hydrophilic, generally the material can have a contact angle of most 90 degrees in some embodiments. In some embodiments the contact angle with water can be no more than 150 degrees. For example, in some embodiments, the contact angle of the hydrophobic material may be in a range of at least 70 degrees to about 120 degrees with an average contact angle of at least 90 degrees, or in a range of at least 120 degrees to 150 degrees. Water contact angles can be measured using any standard apparatus. Although manual goniometers can be used to visually approximate contact angles, contact angle measuring instruments can often include an integrated system involving a level stage, liquid dropper such as a syringe, camera, and software designed to calculate contact angles more accurately and precisely, among other things. Non-limiting examples of such integrated systems may include the FTÅ125, FTÅ200, FTÅ2000, and FTÅ4000 systems, all commercially available from First Ten Angstroms, Inc., of Portsmouth, Va., and the DTA25, DTA30, and DTA100 systems, all commercially available from Kruss GmbH of Hamburg, Germany. Unless otherwise specified, water contact angles herein are measured using deionized and distilled water on a level sample surface for a sessile drop added from a height of no more than 5 cm in air at 20-25° C. and 20-50% relative humidity. Contact angles reported herein represent averages of 5-9 measured values, discarding both the highest and lowest measured values. The hydrophobicity of a material herein may be further enhanced with a hydrophobic coating of other materials, such as silicones and fluorocarbons, and by any means known, such as by coating by the other material (e.g., coated using a liquid that may be subsequently dried on the material) or plasma coated.

A hydrophobic material can be any material having a solubility in water of less than 10 mg/L at standard temperature and pressure. A hydrophilic material can be any material having a solubility in water of 10 mg/L and greater at standard temperature and pressure.

It is important to note that the construction and arrangement of the fire suppression system as shown in the various exemplary embodiments is illustrative only. Although only a few embodiments have been described in detail in this disclosure, many modifications are possible (e.g., variations in sizes, dimensions, structures, shapes and proportions of the various elements, values of parameters, mounting arrangements, use of materials, colors, orientations, etc.). For example, the position of elements may be reversed or otherwise varied and the nature or number of discrete elements or positions may be altered or varied. Accordingly, all such modifications are intended to be included within the scope of the present disclosure. Other substitutions, modifications, changes, and omissions may be made in the design, operating conditions and arrangement of the exemplary embodiments without departing from the scope of the present disclosure.

Additionally, any element disclosed in one embodiment may be incorporated or utilized with any other embodiment disclosed herein. For example, the configuration and construction of dressing 10 of the exemplary embodiment described in at least paragraph [0067] may be incorporated in the dressing 10 of the exemplary embodiment described in at least paragraph [0036]. Although only one example of an element from one embodiment that can be incorporated or utilized in another embodiment has been described above, it should be appreciated that other elements of the various embodiments may be incorporated or utilized with any of the other embodiments disclosed herein.

1. A dressing for a wound comprising:
a body layer having a wound facing side and an environment facing side, the body layer comprising one or more perforations, the one or more perforations having a depth into and/or through the body layer; and
one or more indicators located at least partially in the one or more perforations, the one or more indicators having a wound facing end and an environment facing end, wherein each indicator comprises:
a wicking material configured to draw liquid from the body layer to the environment facing end of the indicator; and
a dye in contact with and/or proximate to the environment facing end of the indicator, the dye configured to change color or opacity in response to contact with liquid drawn by the wicking material;
wherein the color or opacity change indicates a level of fullness of the liquid in the body layer.

2. The dressing of claim 1, wherein the dressing comprises a plurality of the indicators, wherein at least two of the plurality of indicators are capable of drawing liquid from different depths in the body layer and/or configured to indicate different levels of fullness of liquid in the body layer.

3. The dressing of claim 1, wherein one or more indicators consist of the wicking material and the dye.

4. The dressing of claim 1, wherein one or more indicators comprise a core of the wicking material.

5. The dressing of claim 1, wherein the dye is disposed at the environment facing end of the one or more indicators.

6. The dressing of claim 1, wherein the one or more indicators are cylindrical members.

7. The dressing of claim 1, wherein one or more indicators further comprise a spacing material on the wound facing end of the indicator, the spacing material being a material different than the wicking material.

8. (canceled)

9. (canceled)

10. The dressing of claim 1, wherein the body layer is a super-absorbent material.

11. The dressing of claim 1, further comprising a liquid impermeable wound drape configured to substantially cover the body layer and the one or more indicators, the drape capable of attaching to and/or being sealed to tissue surrounding the wound.

12. The dressing of claim 11, wherein the wound drape comprises the dye on a wound facing surface directly adjacent the environment facing end of one or more indicators.

13. The dressing of claim 1, wherein the dressing comprises a plurality of the indicators, wherein at least three of the plurality of indicators are capable of drawing liquid from three different depths in the body layer and/or are configured to indicate at least three different levels of fullness of liquid in the body layer.

14. The dressing of claim 1, wherein the dressing comprises a plurality of the indicators, wherein at least four of the plurality of indicators are capable of drawing liquid from four different depths in the body layer and/or are configured to indicate at least four different levels of fullness of liquid in the body layer.

15. The dressing of claim 1, wherein the dressing comprises a plurality of the indicators and at least two indicators comprise a different amount of the wicking material from each other and are configured to indicate that the body layer is filled with liquid to at least two different levels in the body layer.

16. The dressing of claim 1, wherein the dressing comprises a plurality of the indicators and at least three indicators comprise a different amount of the wicking material from each other and are configured to indicate that the body layer is filled with liquid to at least three different levels of fullness.

17. The dressing of claim 1, wherein the dressing comprises a plurality of the indicators and at least four indicators comprise a different amount of the wicking material from each other and are configured to indicate that the body layer is filled with liquid to at least four different levels of fullness.

18. The dressing of claim 1, wherein a first indicator is configured to change color to indicate that the body layer is filled to a quarter of maximum capacity with liquid;
a second indicator is configured to change color to indicate that the body layer is filled to half of the maximum capacity with liquid;
a third indicator is configured to change color to indicate that the body layer is filled to three quarters of the maximum capacity with liquid; and
a fourth indicator is configured to change color to indicate that the body layer is filled to the maximum capacity with liquid.

19. The dressing of claim 18, wherein three quarters of the first indicator comprises the wicking material, half of the second indicator comprises the wicking material, and a quarter of the third indicator comprises the wicking material.

20. The dressing of claim 1, further comprising an outermost layer, wherein the outermost layer is directly adjacent an environment facing end of at least two indicators, wherein the dye is disposed on a wound facing surface of the outermost layer and is directly adjacent to the environment facing end of the at least two indicators.

21. The dressing of claim 1, wherein the dye comprises hydrochromic ink.

22. (canceled)

23. The dressing of claim 1, further comprising a patient interface layer positioned between the wound and the body layer.

24. The dressing of claim 23, wherein the patient interface layer comprises a foam layer.

25. A dressing for a wound comprising:
a liquid impermeable cover configured to seal to skin surrounding the wound to define an inner volume; and
a plurality of sensors positioned within the inner volume defined by the cover, wherein each of the plurality of sensors are configured to transfer aqueous fluid at a bottom end of the sensor to an upper end of the sensor;
wherein at least one of the sensors and the cover comprises a visual indication portion configured to change color or opacity in response to the sensor transferring aqueous fluid from the bottom end to the upper end, wherein the change of color or opacity indicates a fluid level within the inner volume defined by the cover.

26.-54. (canceled)

55. A method for indicating a liquid level within a wound dressing, the method comprising:
absorbing liquid within an inner volume of the wound dressing;
transferring the liquid through a wicking material to an upper end of the wicking material; and
changing a color or opacity of at least one of the wicking material, a sensor portion adjacent the wicking material, and a portion of a wound drape corresponding to the wicking material in response to contact with the liquid to indicate a liquid level within the wound dressing.

56. (canceled)

57. (canceled)

58. A method of using the dressing of claim 1, the method comprising:
providing the dressing over the wound;
performing negative pressure wound therapy for the wound using a negative pressure wound therapy device fluidly coupled to an inner volume of the wound and/or the dressing; and
monitoring a visual indication of the dressing that indicates an amount of fluid within the dressing.

59. (canceled)

60. A dressing for a wound, comprising:
a body layer formed from an absorbent material; and
a plurality of wicking cores disposed within the body layer, two or more of the wicking cores configured to provide a visual indication at a different state of liquid absorption in the body layer.

61.-91. (canceled)
